(19) United States
(12) Patent Application Publication
(10) Pub. No.: US 2024/0280559 A1
(43) Pub. Date: Aug. 22, 2024

(54) DETERMINING MILD TRAUMATIC BRAIN INJURY, RECOVERY AND TREATMENT

(71) Applicant: LONDON HEALTH SCIENCES CENTRE RESEARCH INC., London (CA)

(72) Inventor: Douglas Dale FRASER, London (CA)

(73) Assignee: LONDON HEALTH SCIENCES CENTRE RESEARCH INC., London, ON (CA)

(21) Appl. No.: 18/571,286

(22) PCT Filed: Jun. 17, 2022
(86) PCT No.: PCT/CA2022/050973

§ 371 Date: Dec. 18, 2023

Related U.S. Application Data
(60) Provisional application No. 63/211,771, filed on Jun. 17, 2021.

(51) Int. Cl. G01N 33/50; A61K 45/06

(52) U.S. Cl. CPC G01N 33/50; A61K 45/06; G01N 2800/28; G01N 2800/52

(57) ABSTRACT

A method of diagnosing mild traumatic brain injury (mTBI) in a subject that includes measuring levels of a biomarker in a test sample; and comparing the levels of the biomarker in the test sample with a known normal reference level of the biomarker, a decrease in the level of the biomarker in the test sample relative to the known normal reference level of the biomarker being indicative of mTBI diagnosis in the subject. The biomarker being one or a combination of two, three, four, five or six of acetic acid, formate, creatine, acetone, methanol, and glutamic acid. Methods of treating mTBI including administering to the subject one or a combination of acetic acid or a source of acetic acid, creatine or a source of creatine, one-carbon metabolism nutrients, a low-carbohydrate diet and/or glutamic acid or a source or precursors of glutamic acid.

### FIELD OF TECHNOLOGY

This disclosure relates to the diagnosis of central nervous system (CNS) injuries.

### BACKGROUND INFORMATION

Acquired central nervous system (CNS) injury (ACNSI), includes acquired brain injury (ABI) and acquired spinal injury (ASI). ABI and ASI can be traumatic and non-traumatic.

Traumatic brain injury (TBI) is an insult to the brain from an external mechanical or pressure-wave force, leading to permanent or temporary impairment of cognitive, physical, and psychosocial functions, with an associated diminished or altered state of consciousness (includes both mechanical and blast injury). The Head Injury Interdisciplinary Special Interest Group of the American Congress of Rehabilitation Medicine defines “mild” TBI as “a traumatically induced physiologic disruption of brain function, as manifested by one of the following: (a) any period of loss of consciousness (LOC); (b) any loss of memory for events immediately before or after the event; (c) any alteration in mental state at the time of the event; and (d) focal neurologic deficits, which may or may not be transient; but where the severity of the injury does not exceed the following: loss of consciousness of approximately 30 minutes or less; after 30 minutes, an initial Glasgow Coma scale of 13-15; and cross post traumatic amnesia no greater than 24 hours period. The Glasgow Coma Scale (GCS) helps defines the severity of a TBI (3-8, severe; 9-12 moderate; 13-15 mild), based on eye, verbal and motor responses. TBI is a major public health concern of epidemic proportions, with an annual incidence of 1.6 to 3.2 million in the United States. According to the Centers for Disease Control and Prevention, National Center for Injury Prevention and Control, mild TBI or mTBI, of which concussion and blast wave injury are subsets, is the most common form, representing nearly 75% of all TBIs. Concussion may be caused by mechanical forces in which the head strikes or is struck by an object, or impulsive forces, in which the head moves without itself being subject to trauma (for example, when the chest hits something and the head snaps forward), or by a pressure wave from a blast. All age groups suffer concussions, from the very young to the elderly. Certain activities are more frequently associated with concussions, including athletics and military service, but they also result from general trauma caused by motor vehicle collisions, falls from height and assaults. Concussions often result in significant acute symptoms and in some individuals, long-term neurological dysfunction.

Blast exposure is common in military service. [1, 2]. A blast wave is generated by an explosion, resulting in a sudden increase in air pressure above ambient conditions (atmospheric pressure) that is followed by negative pressure, or suction of the blast wave. [3] The injury magnitude of a blast wave depends on multiple variables, including the peak of the initial positive-pressure wave, the duration of the overpressure, the medium of the explosion, the distance from the incident blast wave, and the degree of focusing due to a confined area or walls. [4] Military breachers and range staff (MBRS) are exposed to repetitive low-level blasts of 2-3 pounds per square inch (psi; or 14-21 kPa) during training and deployment. [5-8] Larger blast exposure in the range of 60-80 psi (414-522 kPa) are considered lethal. [9]

While the entire body is susceptible to blast injury or blast wave injury, the brain seems particularly susceptible. [4] A blast wave not only reflects off the skull, but the generated energy of the shock wave is also absorbed by the brain tissues. [3] The kinetic injury from thoracoabdominal compression can also result in transmitted forces to the brain via blood vessels. Regardless of the mechanism, poet-blast neurocognitive deficits have been demonstrated in animal models and humans. Repetitive low-level blasts, while unlikely to cause mechanical trauma via acceleration and/or rotation of the head, still result in neurocognitive deficits and general decreased physical and mental health. [7]

Animal models of blast injury have suggested metabolic impairments that include altered glucose metabolism, associated with a shift from aerobic to anaerobic metabolic pathways. Indeed, increased lactate/pyruvate ratio has been reported, followed by decreased energy reserve, [10, 11] oxidative stress and inflammation. [12-16]. The alterations in energy metabolism suggest that blast investigations would benefit from metabolomics profiling, or the measurement of a subject's small metabolite profile, including amino acids, acylcarnitines, glycerophospholipids, sphingolipids, sugars and volatile organic compounds (VOC). Two complementary analytical methods for metabolomics are proton nuclear magnetic resonance (1H NMR) spectroscopy and mass spectrometry (MS), yielding measurement accuracies in the μM and pM ranges, respectively.

While diagnosis of moderate to severe TBI is straightforward, mild TBI is under-diagnosed following concussion and explosive events. [“Blast Injuries: Traumatic Brain Injuries from Explosions”, Brainline.org.] That is, while moderate and severe TBI are easily diagnosed based on clinical signs, mild TBI can be missed due to subtle, transient or absent clinical signs. The latter require an objective diagnostic, such as a blood test that is sensitive, specific and reproducible.

Diagnosis of clinically significant mTBI can be difficult, as are the decisions to stop play, training and/or work activities. For military personnel, it is unclear when to remove the person from training, deployment or theatre of war. It is also unclear when mTBI patients should return to daily activities or active service. Thus, there is great interest in discovery of biomarkers to aid in mTBI, including concussion and primary brain blast injury diagnoses, prognoses, and rehabilitation. At present, no single biomarker has sufficient sensitivity and specificity.

WO 2016/149808 (WO'808), the contents of which are incorporated herein by reference, demonstrates that accurate diagnosis of ACNSI (acquired CNS injury), which includes acquired brain injury and acquired spinal injury, can be aided by metabolomic profiling and machine learning. WO'808 compares a metabolomics profile of an injured subject to what is believed to be a normal cohort of individuals. Accurate diagnosis was obtained by comparing profiles of at least 17 metabolites, but the direction of metabolite changes and the recovery of the subjects was not assessed. The 17 metabolites were measured with two techniques, mass spectrometry (MS) and NMR. They may also need collection devices and different standards for each metabolite.

It would be beneficial to have a small number of diagnostic markers measured with analytical methods, or metabolomics profiling, such as NMR or MS or any other applicable technique that could serve not just to obtain an accurate diagnosis of injuries of the central nervous system, such as concussion and blast wave injury, but also that could serve to follow up the recovery of the patients. It would also be beneficial to have a small number of diagnostic markers whose presence and/or levels can be measured in breath condensate For the latter, breath condensate metabolites can be measured directly by blowing into a breathalyzer or MS, or by blowing into a collecting device for later measurement, such as a balloon or cartridge device.

### SUMMARY OF DISCLOSURE

In one embodiment, the present disclosure is a method of diagnosing mild traumatic brain injury (mTBI) in a subject comprising: (a) obtaining a test sample from the subject; (b) quantitatively or semi-quantitatively measuring levels of a biomarker in the test sample; and (c) comparing the levels of the biomarker in the test sample with a known normal reference level of the biomarker, wherein a decrease in the level of the biomarker in the test sample relative to the known normal reference level of the biomarker is indicative of mTBI diagnosis in the subject, the biomarker being one of acetic acid, formate, creatine, acetone, methanol or glutamic acid or a combination of two, three, four, five or six of acetic acid, formate, creatine, acetone, methanol and glutamic acid. In one embodiment of the method of diagnosing mTBI in a subject, the method further comprises obtaining one or more recovery samples from the subject during the subject's treatment for mTBI, wherein an increase in the levels of the biomarker in the one or more recovery samples relative to the levels of the biomarker obtained in the test sample is indicative of a normalization of the subject.

In another embodiment of the method of diagnosing mTBI in a subject, the method further comprises treating the subject for mTBI only when the levels of the biomarker in the test sample is decreased relative to the known normal reference.

In another embodiment of the method of diagnosing mTBI in a subject, the treatment includes administering to the subject acetic acid, creatine, 1C sources or carbohydrate restriction (i.e., ketosis) or a combination thereof.

In another embodiment of the method of diagnosing mTBI in a subject, the biomarker is one of acetic acid, formate, creatine, acetone, methanol or glutamic acid.

In another embodiment of the method of diagnosing mTBI in a subject, the sample is blood and wherein the biomarker is acetic acid and the known normal reference level is about 29 μM, or the biomarker is formate and the known normal reference level is about 54 μM, or the biomarker is creatine and the known normal reference level is about 23 μM, or the biomarker is acetone and the known normal reference level is about 11 μM, or the biomarker is methanol and the known normal reference level is about 36 μM, or the biomarker is glutamic acid and the known normal reference level is about 37 μM.

In another embodiment of the method of diagnosing mTBI in a subject, the biomarker is a combination of acetone, methanol and glutamic acid.

In another embodiment of the method of diagnosing mTBI in a subject, the biomarker is a combination of creatine, methanol, and glutamic acid.

In another embodiment of the method of diagnosing mTBI in a subject, the biomarker is a combination of acetic acid, acetone and methanol.

In another embodiment of the method of diagnosing mTBI in a subject, the sample is a blood sample, a plasma sample, a serum sample, a capillary sample, a sweat sample, a tear sample, an exhale breath condensate sample or a combination thereof.

In another embodiment of the method of diagnosing mTBI in a subject, the mTBI is primary blast in blast-induced traumatic brain injury or concussion.

In another embodiment, the present disclosure provides a mTBI diagnostic apparatus, the mTBI diagnostic apparatus including a non-transitory computer readable storage medium and a computer program mechanism embedded therein, the computer program mechanism comprising executable instructions for performing a method of diagnosing mTBI in a subject, said executable instructions comprising: (a) receiving quantitative or semi-quantitative levels of a biomarker in a test sample of the subject; (b) comparing the quantitative or semi-quantitative levels of the biomarker in the test sample of the subject to known normal reference levels of the biomarker; and (c) providing a mTBI positive signal when there is a decrease in the level of the biomarker the test sample relative to the known normal reference, the biomarker being one of acetic acid, formate, creatine, acetone, methanol or glutamic acid or a combination of two, three, four, five or six of acetic acid, formate, creatine, acetone, methanol and glutamic acid.

In one embodiment of the mTBI diagnostic apparatus, the biomarker is one of acetic acid, formate, creatine, acetone, methanol, or glutamic acid.

In another embodiment of the mTBI diagnostic apparatus, the sample is blood and wherein the biomarker is acetic acid and the known normal reference level is about 29 μM, or the biomarker is formate and the known normal reference level is about 54 μM, or the biomarker is creatine and the known normal reference level is about 23 μM, or the biomarker is acetone and the known normal reference level is about 11 μM, or the biomarker is methanol and the known normal reference level is about 36 μM, or the biomarker is glutamic acid and the known normal reference level is about 37 μM.

In another embodiment of the mTBI diagnostic apparatus, the biomarker is a combination of acetone, methanol, and glutamic acid.

In another embodiment of the mTBI diagnostic apparatus, the biomarker is a combination of creatine, methanol, and glutamic acid.

In another embodiment of the mTBI diagnostic apparatus, the biomarker is a combination of acetic acid, acetone, and methanol.

In another embodiment of the mTBI diagnostic apparatus, the biomarker is one of acetic acid, acetone or methanol, or a combination of two or more of acetic acid, acetone and methanol, and the apparatus is a breathalyzer, a portable mass spectrometer (MS), or a collection device for laboratory MS measurement.

In another embodiment of the mTBI diagnostic apparatus, the mTBI is primary blast in blast-induced traumatic brain injury.

In another embodiment of the mTBI diagnostic apparatus, the mTBI is concussion.

In another embodiment, the present disclosure provides for a device that measures acetic acid, formate, creatine, acetone, methanol, and glutamic acid.

In one embodiment, the device measures acetic acid, acetone and methanol, and the device is a breathalyzer, a portable MS, or a collection device for laboratory MS measurement.

In another embodiment, the device includes an output signal that turns on or off when the levels of acetic acid, acetone and methanol are below a predetermined cut off value. In aspects, the cut off value is 29 μM for acetic acid, 11 μM for acetone and 36 μM for methanol. In another embodiment, the present disclosure relates to a method for diagnosing and monitoring mTBI in a subject, comprising: (a) collecting exhaled breath condensate from the subject; (b) contacting the exhaled breath condensate with a reaction reagent; and (c) determining whether the subject has mTBI based on a physical change that occurs when the collected exhaled breath condensate contacts the reaction reagent, wherein said physical change is caused when any one of acetic acid, acetone and methanol in the exhaled breath condensate is below or above the cut off values. In one aspect, the cut off values are equivalent to 29 μM of acetic acid, 11 μM of acetone and 36 μM of methanol in blood.

In another embodiment, the present disclosure relates to a method for diagnosing and monitoring mTBI in a subject, comprising: (a) collecting exhaled breath condensate from the subject; (b) measuring the levels of acetic acid, acetone and methanol in the breath condensate; and (c) determining whether the subject has mTBI when the levels of any one of acetic acid, acetone and methanol in the exhaled breath condensate is below the cut off values. In aspects, the cut off values are equivalent to 29 μM of acetic acid, 11 μM of acetone and 36 μM of methanol in blood.

In another embodiment, the present disclosure relates to a method of treating a subject suffering mTBI, wherein the method comprises administering to the subject acetic acid or a source of acetic acid.

In another embodiment, the present disclosure relates to the use of acetic acid or a source of acetic acid in the treatment of mTBI.

In another embodiment, the present disclosure relates to a method of treating a subject suffering mTBI, wherein the method comprises administering to the subject creatine or a source of creatine. In another embodiment, the present disclosure relates to a use of creatine or a source of creatine in the treatment of mTBI.

In another embodiment, the present disclosure relates to a method of treating a subject suffering mTBI, wherein the method comprises administering to the subject one-carbon metabolism nutrients.

In another embodiment, the present disclosure relates to a use of one-carbon metabolism nutrients in the treatment of mTBI.

In another embodiment, the present disclosure relates to a method of treating a subject suffering mTBI, wherein the method comprises administering to the subject a low-carbohydrate diet.

In another embodiment, the present disclosure relates to a use of a low-carbohydrate diet in the treatment of mTBI.

In another embodiment, the present disclosure relates to a method of treating a subject suffering mTBI, wherein the method comprises administering to the subject glutamic acid or precursors of glutamic acid (for example 5-HTP, glutamine and so forth).

In another embodiment, the present disclosure relates to a use of glutamic acid or precursors of glutamic acid (i.e., 5-HTP, glutamine and so forth) in the treatment of mTBI.

In another embodiment, the present disclosure relates to a method of treating a subject suffering mild traumatic brain injury (mTBI), the method comprising administering to the subject one or a combination of two or more of acetic acid, a source of acetic acid, creatine, a source of creatine, one-carbon nutrients, a low carbohydrate diet, glutamic acid, and a precursor of glutamic acid.

In another embodiment, the present disclosure provides for a use of one or a combination of two or more of acetic acid, a source of acetic acid, creatine, a source of creatine, one-carbon nutrients, a low carbohydrate diet, glutamic acid, and a precursor of glutamic acid in the treatment of mild traumatic brain injury (mTBI).

### BRIEF DESCRIPTION OF THE DRAWINGS

The following figures illustrate various aspects and preferred and alternative embodiments of this disclosure.

FIGS. 1A to 1C. Metabolites identified with feature selection that determine military breacher/range staff (MBRS) status and their relationship with Rivermead post-concussion variables. 1A) A tSNE plot demonstrating that MBRS and non-MBRS can be easily separated and identified based on plasma levels of the leading 6 metabolites. The axes are dimension-less. 1B) A heat map demonstrating the negative correlations between Rivermead post-concussion variables and plasma levels of the 6 leading metabolites. Lighter grey represents a stronger negative correlation. Statistically significant negative correlations are indicated with white asterixis. 1C) ROC curves illustrating that the Rivermead post-concussion variables are predictive of MBRS, or blast mTBI, status, as well as with the metabolite parsimonious combinations listed in Table 4 [RPQ13 (late symptoms) AUC=0.79 [0.65-0.94], RPQ3 (early symptoms) AUC=0.77 [95% CI 0.62-0.93], Somatic AUC=0.75 [95% CI 0.58-0.91], Cognitive AUC=0.71 [95% CI 0.53-0.88], and Emotional AUC-0.69 [95% CI 0.52-0.87].

FIGS. 2A to 2F. Leading 6 metabolite measurements plotted against Rivermead RPQ13 (late symptoms) for both MBRS and non-MBRS. Rivermead RPQ13 symptoms are increased in MBRS, and metabolite levels often fall below the established cut off value. 2A—acetic acid, 2B—formate, 2C—creatine, 2D—acetone, 2E—methanol, 2F—glutamic acid.

FIGS. 3A to 3F. Leading 6 metabolite measurements plotted against Rivermead RPQ3 (early symptoms) for both MBRS and non-MBRS. Rivermead RPQ3 symptoms are increased in MBRS, and metabolite levels often fall below the established cut off value. 3A—acetic acid, 3B—formate, 3C—creatine, 3D—acetone, 3E—methanol, 3F—glutamic acid.

FIGS. 4A to 4F. Leading 6 metabolite measurements plotted against Rand Energy scores for both MBRS and non-MBRS. Energy levels are reduced in MBRS, and metabolite levels often fall below the established cut off value. 4A—acetic acid, 4B—formate, 4C—creatine, 4D—acetone, 4E—methanol, 4F—glutamic acid.

### DETAILED DESCRIPTION

### Abbreviations

Unless defined otherwise, all technical and scientific terms used herein have the same meaning as commonly understood by one of ordinary skill in the art to which this disclosure belongs. Also, unless indicated otherwise, except within the claims, the use of “or” includes “and” and vice versa. Non-limiting terms are not to be construed as limiting unless expressly stated or the context clearly indicates otherwise (for example “including”, “having” and “comprising” typically indicate “including without limitation”). Singular forms including in the claims such as “a”, “an” and “the” include the plural reference unless expressly stated otherwise. “Consisting essentially of” means any recited elements are necessarily included, elements that would materially affect the basic and novel characteristics of the listed elements are excluded, and other elements may optionally be included. “Consisting of” means that all elements other than those listed are excluded. Embodiments defined by each of these terms are within the scope of this disclosure.

All numerical designations, e.g., levels, amounts and concentrations, including ranges, are approximations that typically may be varied (+) or (−) by increments of 0.1, 1.0, or 10.0, as appropriate. All numerical designations may be understood as preceded by the term “about”.

“Baseline” means a measurement of reference of a single metabolite level or multiple metabolite levels in a subject before an event that produces an acquired central nervous system (CNS) injury in the subject, or prior to the start of an activity, such as prior to a sports season or military training.

In this document, references to glutamic acid (symbol Glu or E) include glutamic acid and its ionic form glutamate.

In this document the definition of “mild traumatic brain injury”, “mTBI””, which may also be referred to in the literature as mild head injury or concussion, is that taken from the American Congress of Rehabilitation Medicine (ACRM; J Head Trauma Rehabil 1993; 8(3):86-87), and it refers to a person who has had a traumatically induced physiological disruption of brain function, as manifested by at least one of the following: 1) any period of loss of consciousness; 2) any loss of memory for events immediately before or after the event; 3) any alteration in mental state at the time of the event (e.g., feeling dazed, disoriented, or confused); and 4) focal neurological deficit(s) that may or may not be transient; but where the severity of the injury does not exceed the following: loss of consciousness of approximately 30 minutes or less; after 30 minutes, an initial Glasgow Coma Scale (GCS) of 13-15; and posttraumatic amnesia (PTA) not greater than 24 hours. This definition includes: 1) the head being struck, 2) the head striking an object, and 3) the brain undergoing an acceleration/deceleration movement (i.e., whiplash) without direct external trauma to the head. For this use, a pressure wave caused by a blast is also a recognized cause of mTBI, or concussion. Computed tomography, magnetic resonance imaging, electroencephalogram, near infrared spectroscopy, positive emission tomography or routine neurological evaluations may be normal. Due to the lack of medical emergency, or the realities of certain medical systems, some patients may not have the above factors medically documented in the acute stage. In such cases, it is appropriate to consider symptomatology that, when linked to a traumatic head injury, can suggest the existence of a mTBI.

“Metabolome” refers to the collection of all metabolites in a biological cell, tissue, organ or organism, which are the end products of cellular processes. Metabolites in the metabolome may be endogenous (produced and metabolized by the individual themselves) or exogenous (obtained from external sources and metabolized within the individual, either by host metabolism or microflora) in origin. Metabolites may be altered by lifestyle factors and the microbiome. “Metabolome” includes lipidome, sugars, nucleotides and amino acids. Lipidome is the complete lipid profile in a biological cell, tissue, organ or organism.

“Metabolomic profiling” refers to the characterization and/or measurement of the small molecule metabolites in biological specimen or sample, including cells, tissue, organs, organisms, or any derivative fraction thereof and fluids such as blood, blood plasma, blood serum, capillary blood, venous blood, saliva, synovial fluid, spinal fluids, urine, bronchoalveolar lavage, tissue extracts, tears, breath samples, sweat, and so forth. This characterization may be targeted (limited to a defined number of specific compounds) or untargeted/nontargeted in nature (not limited to a defined or known number of compounds).

The metabolite profile may include information such as the quantity and/or type of small molecules present in the sample. The ordinarily skilled artisan would know that the information which is necessary and/or sufficient will vary depending on the intended use of the “metabolite profile.” For example, the “metabolite profile,” can be determined using a single technique for an intended use but may require the use of several different techniques for another intended use depending on such factors as the disease state involved, the types of small molecules present in a particular targeted cellular compartment, the cellular compartment being assayed per se., and so forth.

The relevant information in a “metabolite profile” may also vary depending on the intended use of the compiled information, e.g., mass spectrum or chromatogram. For example, for some intended uses, the amounts of a particular metabolite or a particular class of metabolite may be relevant, but for other uses the distribution of types of metabolites may be relevant.

Metabolite profiles may be generated by several methods, e.g., liquid chromatography (LC), high performance LC (HPLC), ultra-high performance LC (UHPLC), ultra-performance LC (UPLC), thin layer chromatography (TLC), electrochemical analysis, Mass Spectrometry (MS), tandem Mass Spectrometry (MS/MS), Proton Transfer Reaction-Mass Spectrometry (PTR-MS), time-of-flight mass spectrometry (TOF-MS), selected Ion Flow Tube technique, refractive index spectroscopy (RI), Ultra-Violet spectroscopy (UV), fluorescent analysis, radiochemical analysis, Near-InfraRed spectroscopy (Near-IR), Nuclear Magnetic Resonance spectroscopy (NMR), fluorescence spectroscopy, dual polarization interferometry, flame ionization detection (FID), computational methods, =Light Scattering analysis (LS), gas chromatography (GC), or GC coupled with MS, direct injection (DI) coupled with MS/MS and/or other methods or combination of methods known in the art.

The term “subject” as used herein refers all members of the animal kingdom including mammals, preferably humans.

The term “patient” as used herein refers to a subject that is suspected of having mild traumatic brain injury (mTBI), mTBI includes concussion and blast injuries, including blast overpressure wave injury.

### Overview

The present disclosure relates to the use of at least one specific metabolite/biomarker or a cohort (group or a combination of more than 1) of up to six specific metabolites/biomarkers to accurately diagnose mTBI, including blast injury. Treatment of mTBI may include rest, symptomatic treatment and supportive care, followed by gradual return to activities. It is imperative to protect against a repeat blat injury, particularly while healing. There are symptom therapies (i.e., drugs for headaches, anxieties and depression, and sleep disturbances, as well as educational and cognitive support) and rehabilitation steps, including neck strengthening, aerobic exercise, massage, etc.).

In one embodiment, the approach of the present disclosure involves obtaining a sample from the subject at baseline (i.e., before the subject suffers a mTBI injury), shortly after a suspected mTBI injury, and again during the recovery period. Up to 6 specific metabolites (i.e., 1 metabolite or groups of 2, 3, 4 5 or 6 metabolites) are measured in the samples taken from the same subject at the different times (baseline, post-injury and during recovery) are compared using quantitative or semi-quantitative measurements (baseline vs. after suspected injury), and again during recovery to show normalization of metabolite levels. The subject samples include blood, blood plasma, blood serum, capillary blood/plasma, venous blood, saliva, synovial fluid, urine, spinal fluid, bronchoalveolar lavage, sweat, tears, breath samples (i.e., VOCs) and extracts (for example extracts of hippocampal tissue or ipsilateral cortex tissue). A drop in the level of the at least one of the metabolite species in the post-injury sample being indicative of mTBI. The approach of this embodiment is useful of mTBI including those instigated by mechanical, blast, chemical and psychological trauma. Rather than looking at predefined patterns of metabolites, this embodiment focuses on a change in a single metabolite species or changes in a cohort of up to 6 specific metabolite species. This embodiment is advantageous for individuals whose baseline blood/plasma, capillary blood/plasma samples can be obtained because they are about to take part in an activity with risk of injury, such as sports and military service. It may also be conceivable that insurance companies may require athletes as well as subjects involved in specific activities, such as soldiers, police, firefighters, construction workers, miners, drivers, to take baseline samples before granting insurance to those subjects.

In another embodiment, the present disclosure involves comparing the levels of a single specific metabolite species or a cohort of up to 6 specific metabolite species in a subject's sample, such as blood, blood plasma, blood serum, capillary blood/plasma, venous blood, saliva, synovial fluid, urine, spinal fluid, bronchoalveolar lavage, sweat, tears, breath samples (i.e. VOCs) and extracts (for example extracts of hippocampal tissue or ipsilateral cortex tissue), using quantitative measurements of said single metabolite species or cohort of up to 6 specific metabolite species to the levels of said single metabolite species or cohort of up to 6 metabolite species in a known reference range, cut-off value, or in library of measurements of known mTBI (positive control), or in a library of measurements of known non-mTBI (negative or normal control) (see below for a description of the control library of measurements). A drop in the level of the at least one metabolite species in the subject's sample relative to the known reference range, cut-off value or the negative control population being indicative of the subject having mTBI.

The methods and computer programs of the present disclosure may be used in point-of-care metabolomics testing with portable, table/countertop, wearable devices (i.e., smart watches) or handheld instruments that generate metabolite measurements/profiles.

### Single or Cohort of Up to 6 Metabolite Species

In one embodiment, the single metabolite species and the cohort of up to six metabolite species are from the species listed in Table 3, that is: acetic acid, formate, creatine, acetone, methanol and glutamic acid.

In one embodiment, the cohort of metabolite species include no more than 6 metabolite species. In another embodiment, the cohort of metabolite species include no more than 5 metabolite species. In another embodiment, the cohort of metabolite species include no more than 4 metabolite species. In another embodiment, the cohort of metabolite species include no more than 3 metabolite species. In another embodiment, the cohort of metabolite species include no more than 2 metabolite species.

In one embodiment, the single metabolite species is acetic acid. In another embodiment, the single metabolite species is formate, in another embodiment, the single metabolite species is creatine. In another embodiment, the single metabolite species is acetone. In another embodiment, the single metabolite species is methanol. In another embodiment, the single metabolite species is glutamic acid.

In one embodiment of the present disclosure, the cohort or combination of metabolite species is acetic acid, methanol and glutamic acid. In another embodiment of the present disclosure, the cohort or combination of metabolite species is acetone, methanol and glutamic acid. In another embodiment of the present disclosure, the cohort or combination of metabolite species is creatine, methanol and glutamic acid. In another embodiment of the present disclosure, the cohort or combination of metabolite species easily measured in breath, such as acetic acid, acetone and methanol.

Since metabolites exist in a very broad range of concentrations and exhibit chemical diversity, there is no one instrument that can reliably measure all of the metabolites in the non-human or human metabolome in a single analysis. Instead, practitioners of metabolomic profiling generally use a suite of instruments, most often involving different combinations of liquid chromatography (LC) or gas chromatography (GC) coupled with MS, to obtain broad metabolic coverage [Circulation. 2012; 126: 1110-1120]. Other instruments such as NMR, electrochemical analysis, RI, UV, near-IR, LS, GC coupled to non-MS detectors and so forth may also be used.

Point-of-care testing (e.g., tabletop MS, hand-held breathalyzer) could be developed to identify ACNSI, including mTBI and non-TBI patients, and to prognosticate their brain injuries.

A library of measurements of the metabolites listed in Table 3 may be established for diagnosed mTBI cases. For example, a library of measurements of the metabolites listed in Table 3 for concussion and for primary blast in blast-induced traumatic brain injury and any other possible form of mTBI. This library may be used as the predetermined, control set of the metabolite measurements of mTBI (referred to as positive control). A comparison may be made of the subject's metabolic species measurements against the predetermined metabolic species measurements of mTBI and the predetermined metabolite species measurements of non-mTBI (referred to as negative control or normal control) to determine not only if the patient has mTBI, but also the type of mTBI (i.e. concussion, primary blast in blast-induced traumatic brain injury, electrical-induced brain injury (electrocution), seizure-induced injury, surgical-induced injury, stroke-induced injury, poison-induced injury, psychological injury, chemical injury, infectious injury, ischemic injury, metabolic injury, inflammatory injury, autoimmune injury, degenerative injury, hypoxic injury, and cancer/radiation-induced injury and so forth) and the prognosis.

The libraries of predetermined metabolite species measurements (mTBI control library and normal control libraries) may be provided in a computer product (memory sticks, as an app for handheld devices such as tablets, pads, smart watches, cellular phones and so forth), or they may be uploaded to the memory of a computer system, including main frames, desktops, laptops, handheld devices such as tablets, pads, smart watches and cellular phones. Blood or any other bodily fluid, for example whole blood, blood plasma, blood serum, capillary blood sample, saliva, synovial fluid, urine, spinal fluid, bronchoalveolar lavage, tears, sweat, extracts, breath sample (e.g., VOCs) and so forth, may be taken from a subject suspected of having an ABI and/or ASI. The VOCs are emitted from certain solids or liquids, for the latter, the VOCs can be measured from breath or breath condensate. A device for collecting and quantitatively analyzing vapor condensate (VC) sample can be used to collect and measure the 3 VOCs, i.e., acetic acid, acetone and methanol. Breath condensate metabolites can be measured directly by blowing into a breathalyzer or MS, or by blowing into a collecting device for later measurement, such as a balloon or cartridge device. In one embodiment, the device for measuring the volatile organic compounds acetic acid, acetone and methanol includes an output signal that turns on or off when the levels of acetic acid, acetone and methanol are below a cut off value thereby indicating a positive mTBI diagnosis. For breathalyzers that measure in parts per billion (PPB), the cut off values are equivalent to 29 μM, 11 μM and 36 μM respectively in blood. In another embodiment, the device also includes reaction reagents that physically change, for example a change in color of the reaction reagent, upon contact with the breath condensate when the breath condensate includes normal levels acetic acid, acetone and methanol (i.e., levels above the cut off values of 29 μM, 11 μM and 36 μM) thereby providing a negative mTBI diagnosis, but will not physically change (for example no change in color of the reaction reagent), when the levels of acetic acid, acetone and methanol are below the normal levels (i.e., levels below the cut off values of 29 μM, 11 μM and 36 μM for acetic acid, acetone and methanol respectively), thereby giving a positive mTBI diagnosis.

Alternatively, the reaction reagent may physically change when the levels of the acetic acid, acetone and methanol are below the previously listed cut off values thereby providing a positive mTBI diagnosis.

As such, the present disclosure provides also for a point-of-care method for diagnosing and monitoring mTBI in a subject, comprising: (a) collecting exhaled breath condensate from the subject; (b) contacting the exhaled breath condensate with a reaction reagent; and (c) determining whether the subject has mTBI based on a physical change that occurs when the collected exhaled breath condensate contacts the reaction reagent, wherein said physical change is caused when any one of acetic acid, acetone and methanol in the exhaled breath condensate is below or above the cut off values, wherein the cut off values are equivalent to 29 μM of acetic acid, 11 μM of acetone and 36 μM of methanol in blood.

The present disclosure provides also for a method for diagnosing and monitoring mTBI in a subject, comprising: (a) collecting exhaled breath condensate from the subject; (b) measuring the levels of acetic acid, acetone and methanol in the breath condensate; and (c) determining whether the subject has mTBI when the levels of any one of acetic acid, acetone and methanol in the exhaled breath condensate is below the cut off values, wherein the cut off values are equivalent to 29 μM of acetic acid, 11 μM of acetone and 36 μM of methanol in blood. The device may contain algorithms to integrate results from multiple VOCs.

Blood samples can be obtained using traditional blood draws or collected via capillary prick on to dried plasma spots (DPS). In one embodiment, capillary blood is drawn from a puncture made on a finger using an incision device, such as a lancet or any other incision device [Lenicek Krleza J, Dorotic A, Grzunov A, Maradin M. Capillary blood sampling: National recommendations on behalf of the Croatian society of medical biochemistry and laboratory medicine. Biochem Medica. 2015; 25:335-58]. Blood is then applied to a blood collection device which is or contains a paper material (such as cellulose filter paper, a glass fibre membrane or other suitable materials). Blood may be applied to the device directly from the finger via a hanging drop or via a small pipette or capillary applicator. The device filters out red blood cells from the collected whole blood and permit plasma or serum to flow or soak into the paper collection material. This paper material is air dried and stored in an appropriate manner until extraction of the lipid species and analysis. Metabolite species measurements may be obtained from the subject's sample using any known technology (for example, high performance liquid chromatography, thin layer chromatography, electrochemical analysis, mass spectrometry (MS), refractive index spectroscopy, ultra-violet spectroscopy, fluorescent analysis, radiochemical analysis, near-infrared spectroscopy, light scattering analysis, gas chromatography (GC), or GC coupled with MS, direct injection (DI) coupled with MS/MS, LC coupled with MS/MS and so forth). The subject's metabolite measurements may then be uploaded to the computer system (main frames, desktops, laptops, handheld devices such as phones, tablets, pads, watches, and so forth). An operator may then compare the subject's metabolite species measurements with the predetermined set of metabolite species measurements of mTBI and the predetermined metabolite species measurements of non-mTBI (referred to as “control” or “normal”) to determine not only if the patient has mTBI, but also the type of mTBI, or whether a treatment is efficient.

Optimization of biomarker collection via dried plasma spot (DPS). DPS can be easily sampled at home quickly, safely and less invasively than traditional blood draws, thereby allowing for repeat samples to be taken over the course of a season. However, the choice of plasma collection device is crucial to ensure a good quality, stable and quantitative sample of plasma that is easy and painless to procure. Reproducibility of sampling volume is determined by collecting replicate DPS, extracting the metabolites with a suitable solvent, and comparing the precision of quantification of said metabolites for multiple DPS from various manufacturers. A coefficient of variation (CV) of repeated measurements of metabolites using a device must be <15%. Extraction efficiency and potential matrix effects from the devices is determined by comparing concentrations of neat standard solutions of the metabolites to the metabolites extracted from the DPS. Ideal recovery is 80-120% of each metabolite, however, if extraction shows good reproducibility (i.e. <15% CV) it is acceptable. Stability of the compounds on the dried plasma cards is ascertained by collecting and storing cards under various conditions (i.e. room temperature, 4° C. and −20° C.) and quantifying GPLs over days, weeks and months to determine the extent of degradation of the analytes related to the specific filter paper in the collection device.

Returns to a normal level of the metabolite species may serve as an aid in following medical interventions (including rehabilitation therapy) of individuals affected by mTBI and guide return to work and/or daily activities.

As such, in another embodiment, the present disclosure is a method of tracking or following the efficiency of a medical intervention (including rehabilitation therapy) in a mTBI patient, the method including: (a) obtaining the levels of a metabolite species or a cohort of (i.e., multiple, group or a combination of more than 1) metabolite species from the patient at different times during the medical intervention (including rehabilitation therapy); and (b) using quantitative or semiquantitative measurements for comparing the levels of the patient's single metabolite species or cohort of up to 6 metabolite species during or at each of the different times to a predetermined set of metabolite profiles of mTBI (positive control) and/or to a predetermined set of the single metabolite species or cohort of up to 6 metabolite species of non-mTBI (negative control) to follow the efficiency of the medical intervention in the patient. A return to a normal level of the single metabolite species or cohort of up to 6 metabolite species of the patient relative to the negative control may serve to assess whether the medical intervention (including rehabilitation therapy) of the patient has been successful.

The one or more metabolite(s)/biomarker(s) of the present disclosure may be used to prognosticate the duration of the mTBI therapy and to determine when a patient has normalized. This can be done by following the levels of one or more of the metabolites/biomarkers listed in Table 3 throughout the therapeutic treatment of the patient. Patients may suffer serious outcomes from withdrawal of care after there has been no improvement in in their mTBI. These biomarkers help determine who will have a bad outcome earlier and aid end of life decision making or determine whom will do well and guide persistent management of the mTBI. For example, a concussed brain or blast injured brain is in a vulnerable state that places it at increased risk of more debilitating injury should more trauma occur before metabolic homeostasis is restored. Accordingly, the present disclosure provides for a method of determining when metabolic homeostasis may have been restored in a patient who suffered mTBI.

In another embodiment, this disclosure also provides a kit for use in the methods described herein containing one or more reagents for use in the detection of the one or combination of up to 6 metabolites in a biological sample according to the methods of the present disclosure, and instructions for use. The instructions may also include instructions to treat the subject upon a diagnosis of mTBI.

In another embodiment, this disclosure also provides for a device that measures acetic acid, formate, creatine, acetone, methanol and glutamic acid. In one aspect, the device measures acetic acid, acetone and methanol, and the device is a breathalyzer, a portable MS or a collection device for laboratory MS measurement. In another aspect, the device includes an output signal that turns on or off when the levels of acetic acid, acetone and methanol are below a predetermined cut off value. In aspects, the cut off value is 29 μM for acetic acid, 11 μM for acetone and 36 μM for methanol.

In another embodiment, this disclosure also provides for method for diagnosing and monitoring mild traumatic brain injury (mTBI) in a subject, comprising:
(a) collecting exhaled breath condensate from the subject;
(b) contacting the exhaled breath condensate with a reaction reagent; and
(c) determining whether the subject has mTBI based on a physical change that occurs when the collected exhaled breath condensate contacts the reaction reagent, wherein said physical change is caused when any one of acetic acid, acetone and methanol in the exhaled breath condensate is below or above the cut off values, wherein the cut off values are equivalent to 29 μM of acetic acid, 11 μM of acetone and 36 μM of methanol in blood.

In another embodiment, this disclosure also provides for a method for diagnosing and monitoring mild traumatic brain injury (mTBI) in a subject, comprising:
(a) collecting exhaled breath condensate from the subject;
(b) measuring the levels of acetic acid, acetone and methanol in the breath condensate; and
(c) determining whether the subject has mTBI when the levels of any one of acetic acid, acetone and methanol in the exhaled breath condensate is below the cut off values, wherein the cut off values are equivalent to 29 μM of acetic acid, 11 μM of acetone and 36 μM of methanol in blood.

In another embodiment, the present disclosure provides for a method of treating a subject suffering mild traumatic brain injury (mTBI), wherein the method comprises administering to the subject acetic acid or a source of acetic acid.

In another embodiment, the present disclosure provides for a method of treating a subject suffering mild traumatic brain injury (mTBI), wherein the method comprises administering to the subject creatine or a source of creatine.

In another embodiment, the present disclosure provides for a method of treating a subject suffering mild traumatic brain injury (mTBI), wherein the method comprises administering to the subject one-carbon metabolism nutrients.

In another embodiment, the present disclosure provides for a method of treating a subject suffering mild traumatic brain injury (mTBI), wherein the method comprises administering to the subject a low-carbohydrate diet.

In another embodiment, the present disclosure provides for a method of treating a subject suffering mild traumatic brain injury (mTBI), wherein the method comprises administering to the subject glutamic acid/glutamate or precursors of glutamic acid/glutamate (i.e., 5-HTP, glutamine and so forth).

In another embodiment, the present disclosure provides for a use of one or a combination of two or more of acetic acid, a source of acetic acid, creatine, a source of creatine, one-carbon nutrients, a low carbohydrate diet, glutamic acid and a precursor of glutamic acid/glutamate in the treatment of mild traumatic brain injury (mTBI).

### Therapies

In one embodiment, the present disclosure provides a method for treating mTBI in a subject by administering to the subject one or more of acetic acid, creatine and one-carbon (1C) sources, as well as through carbohydrate restriction (i.e., ketosis), alone or in combination with other agents or therapies.

The levels of acetic acid have been found to be reduced in patients with mTBI relative to the normal control subjects. Administration of acetic acid, for example through supplementation with dietary acetic acid, serves to treat a subject of mTBI.

The levels of creatine in patients with mTBI have also been found to be reduced relative to the normal control subjects. Administration of creatine, for example through creatine dietary supplementation, serves to treat a subject of mTBI.

The levels of formate, an intermediate in one-carbon (1C) metabolism, and methanol have been found to be reduced in patients with mTBI relative to the normal controls. Administration of 1C sources, such as through dietary supplementation, including formate, creatine, choline and betaine, serves to treat a subject of mTBI.

The levels of acetone have been found to be reduced in patients with mTBI relative to the normal controls. Controlling the carbohydrate intake of a subject with mTBI, such as through low-carb diet (keto diet) serves to treat a subject of mTBI.

The levels of glutamic acid have been found to be reduced in patients with mTBI relative to the normal controls. A glutamic acid/glutamate containing diet, or a diet containing a precursor of glutamic acid/glutamate, including 5-HTP and glutamine, may serve to treat a subject suffering mTBI.

As such, in one embodiment, this disclosure provides a method for treating mTBI in a subject by administering to the subject the acetic acid, creatine, glutamic acid and 1C sources, as well as carbohydrate restriction (i.e., ketosis) or a combination thereof.

In another embodiment, the present disclosure provides for a mild traumatic brain injury (mTBI) diagnostic apparatus, the mTBI diagnostic apparatus including a non-transitory computer readable storage medium and a computer program mechanism embedded therein, the computer program mechanism comprising executable instructions for performing a method of diagnosing mTBI in a subject, said executable instructions comprising:
(a) receiving quantitative or semi-quantitative levels of a biomarker in a test sample of the subject;
(b) comparing the quantitative or semi-quantitative levels of the biomarker in the test sample of the subject to known normal reference levels of the biomarker; and
(c) providing a mTBI positive signal when there is a decrease in the level of the biomarker the test sample relative to the known normal reference, the biomarker being one of acetic acid, formate, creatine, acetone, methanol or glutamic acid or a combination of two, three, four, five or six of acetic acid, formate, creatine, acetone, methanol and glutamic acid.

In order to aid in the understanding and preparation of the within disclosure, the following illustrative, non-limiting, examples are provided.

### Examples

As the consequences of long-term exposure to repetitive blasts is largely unknown, as are the injury thresholds, or when blast exposure initiates poorer health and compromised well-being, identification of accurate blast injury biomarkers and assays is critical to understanding blast exposure pathophysiology. Thus, the aims of this study were (1) to profile 2 Canadian Armed Forces (CAF) military member cohorts (Military Breachers/Range Staff [MBRS] and non-MBRS) with metabolomics, (2) to identify novel metabolite biomarkers of MRRS with machine learning; and (3) to correlate biomarkers with blast injury symptoms.

### Methods

The study protocol was approved by the Human Research Ethics Committee of Defence Research and Development Canada. Potential participants were recruited via an electronic recruitment poster that was circulated among Canadian Forces School of Military Engineering (CFSME) staff (MBRS) and at Denison Armory (for non-MBRS or controls).[7] Blast quantification was not attempted; however, the instructors and range staff potentially contribute to 8-20 breaching courses per year with 1-2 days of breaching on the range. The instructors and range staff form a “cell” that administers the courses together for a period of 1-3 years. While they may be exposed to more than 6 blasts per day, the magnitude and number of blast events varies.

All data were collected in a single session for each participant. CFSME MBRS were tested at Canadian Forces Base Gagetown (CFB Gagetown). Sex- and age-matched CAF controls were tested at DRDC (Toronto Research Center). The measures included the neuropsychological and neurocognitive tasks, as well as blood procurement for biomarker studies. Specifically, participants completed a demographic and service history, a Background Health Questionnaire, the RAND SF-36 Health Survey, the Short Musculoskeletal Function Questionnaire, a modified version of the Rivermead Post Concussion Symptoms Questionnaire, a Post-Traumatic Checklist (PCL-5) and a Patient Satisfaction Questionnaire (PSQ).

All subjects had 20 mL of blood drawn into EDTA Vacutainer tubes. No restrictions were placed on the time-of-day collection by intent and design, to better represent the natural state of the subject. Blood was centrifuged and plasma aliquoted into cryovials at a volume of 500 uL and stored at −80ºC. Freeze/thaw cycles were avoided. Plasma was collected by strict standard operating procedures, with equal processing times between cohorts. A targeted quantitative approach was applied to analyze plasma samples using a both 1H NMR and a combination of direct injection MS (DI-MS; AbsoluteIDQTM Kit) with a Liquid Chromatography tandem MS Kit (LC-MS/MS; BIOCRATES Life Sciences AG, Innsbruck, Austria), as previously described.[18]

For feature selection, the raw data for each subject were ingested within each feature, across subjects. A random forest classifier was trained on the variables to predict MBRS status (“scikit-learn” module for Python 3.8.5 Open Source). A random forest is a set of decision trees, and consequently, we were able to interrogate this collection of trees to identify the features that have the highest predictive value. Feature selection was not performed in preprocessing. During training, the random forest classifier performed an implicit feature selection; the top features are those that appear highest ranked in the most trees. To reduce overfitting, the number of trees and maximum depth of each tree was limited; thus, MBRS status was determined using a six-fold cross validation with a random forest of 10 trees. To remain conservative and to limit the risk of overfitting further, no hyperparameters were tuned or optimized by design and intent. The reduced metabolite dataset was then visualized with a nonlinear dimensionality reduction on the full data matrix using the t-distributed stochastic nearest neighbor embedding (t-SNE) algorithm.[20] t-SNE assumes that the “optimal” representation of the data lies on a manifold with complex geometry, but with low dimension, embedded in the full-dimensional space of the raw data.

Medians (IQRs) and frequency (%) were used to report continuous and categorical variables, respectively. Continuous variables were compared using Mann-Whitney tests and categorical variables were compared using chi-square tests (or Fisher's exact chi-square, as appropriate). Receiver operating characteristic (ROC) curves were estimated for individual metabolites and continuous outcomes in terms of predicting MBRS status, with area-under-the-curve (AUC)>0.7 considered acceptable. The coordinates of the curves for individual metabolites were analyzed using Youden's index to identify cutoff values in μM based on the highest sensitivity and specificity for predicting MBRS status. Metabolite combinations were calculated through logistic regression models with MBRS status as the outcome and the representative metabolites as the included predictors; the predicted values from the regression models were then saved for use in ROC curve analyses. All analyses were conducted using SPSS version 27 (IBM Corp., Armonk, NY, USA), and p-values<0.05 were considered significant. Heat maps depicting Pearson correlation values between metabolites and outcomes were created in R (http://www.r-project.org) using the ggplot2 version 3.3.3 package.[21]

### Results

We prospectively included 19 MBRS and 19 age- and sex-matched non-MBRS (Table 1); the final MBRS cohort number was 18 as there was insufficient plasma from one service member for analyses. Nonetheless, the two cohorts were well-balanced for age, sex, education, military status, lifestyle and injuries. The MBRS cohort was more likely to be francophone, senior rank, longer duration of service and exposed to blasts. As expected, MBRS were significantly exposed to a greater number of blasts.

The reported symptoms and health assessment data is listed in Table 2. The MBRS cohort reported an increased number of symptoms, with reduced general physical and mental health. Energy was lower for MBRSs, and function and emotional health suffered. The Rivermead post-concussion symptoms were worse for MBRSs, including earl and late symptoms, as well as somatic, cognitive and emotional health. Finally, perceived stress was significantly worse for MBRSs.

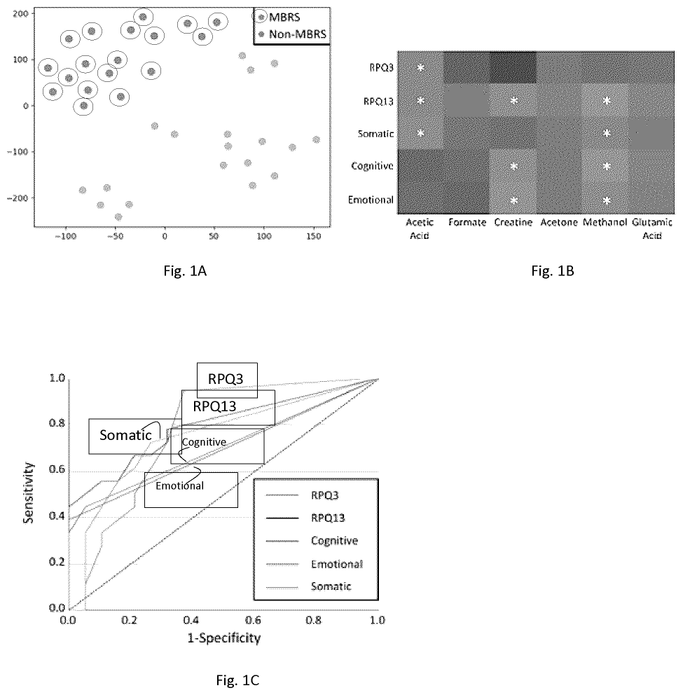

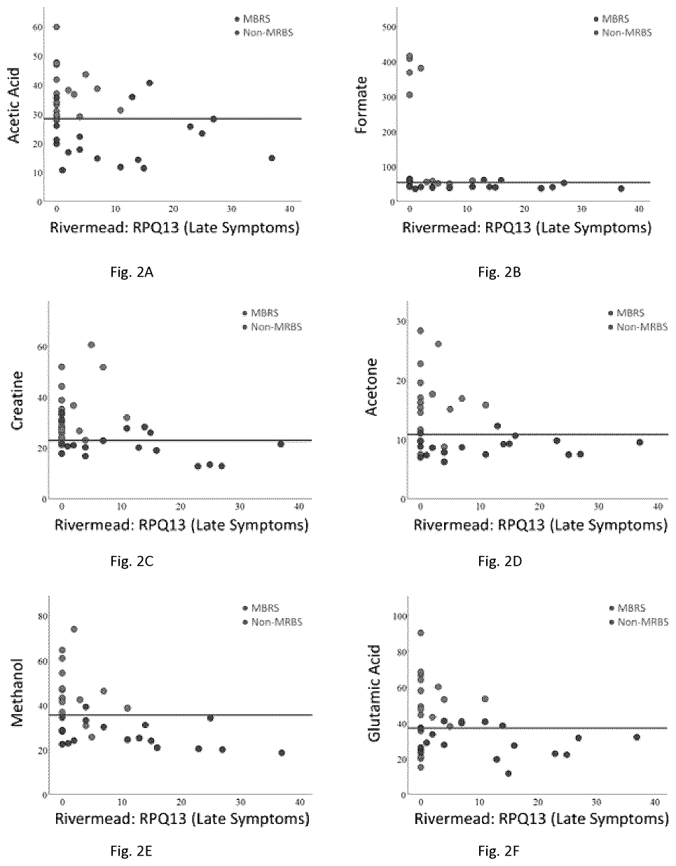

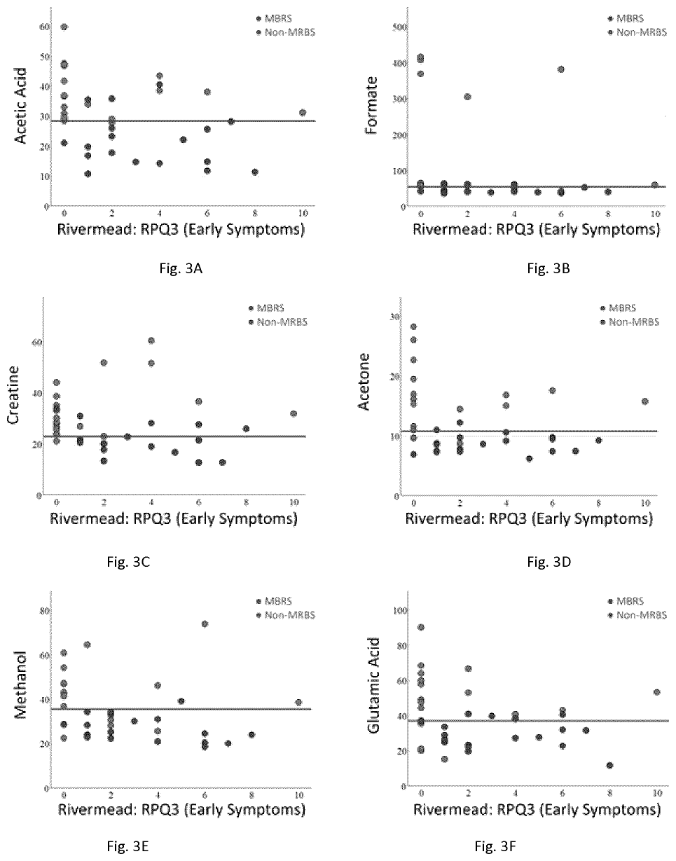

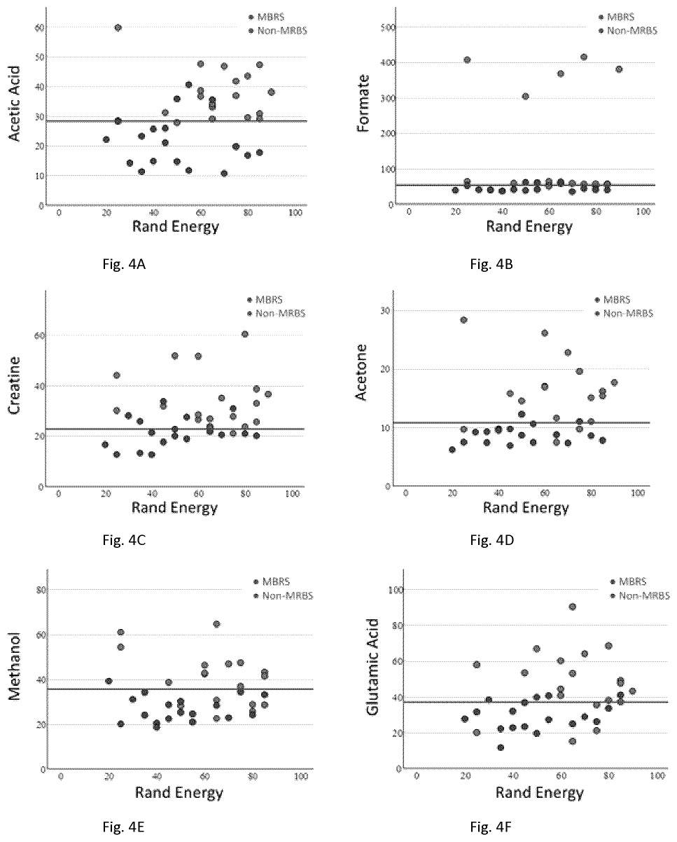

Metabolomics profiling of cohorts was accomplished with both 1H NMR and DI-MS, with a total of 170 plasma metabolites measured. Cohort classification accuracy was 74% when the entire metabolome was ingested. Feature selection narrowed the leading metabolites down to 6, providing a classification accuracy of 98% (Table 5). A rank order of 6 leading plasma metabolites that classify MBRS versus non-MBRS with 98% classification accuracy is listed in Table 5. All 6 metabolites are significantly decreased in plasma from MBRS when compared to non-MBRS. Their relative % importance is shown in Table 5. The 6 metabolites included acetic acid, formate, creatine, acetone, methanol and glutamic acid, and all 6 were all significantly lower in the MBRS cohort (P<0.001; Table 3). A tSNE plot demonstrated near perfect separation of cohorts based on the leading 6 metabolites (FIG. 1A). The decreased levels of acetic acid, creatinine and methanol correlated with increased symptom scores reported on the Rivermead post-concussion questionnaire (FIG. 1B). ROC curve analyses of the 6 individual metabolites for determining MBRS status demonstrated AUCs of 0.82-0.91 (P<0.001; Table 4). The cut-off values for each metabolite was determined. Scatter plots with metabolite cut-off values are shown for Rand energy, as well as Rivermead early (RPQ3) and late (RPQ13) symptoms (FIGS. 2 to 4). We then identified 3 parsimonious combinations of 3 metabolites that perfectly predicted MBRS status with AUCs=1.00 (P<0.001; Table 4). One metabolite combination that consisted of only VOCs, including acetic acid, acetone and methanol, yielded an AUC of 0.98 (P<0.001). The RAND energy level and the Rivermead post-concussion symptom scores predicted both MBRS status and the parsimonious metabolite combinations, yielding AUCs of 0.69-0.79 (FIG. 1C; the Rand energy level not shown, AUC=0.73 [95% CI 0.56-0.90]).

TABLE 1
Military personnel demographics, service
history and injuries/exposures

| | Non-MBRS | | MBRS | | |
|---|---|---|---|---|---|
| Variable | (n = 19) | | (n = 18) | | P-value |
| Age (yrs), med (IQR) | 32 | (27, 36) | 32 | (26, 38) | 0.976 |
| Male sex, n (%) | 17 | (90) | 16 | (89) | >0.994 |
| Education, n (%) | | | | | 0.633 |
| High school | 4 | (21) | 6 | (33) | |
| College | 4 | (21) | 6 | (33) | |
| Undergraduate university | 10 | (53) | 5 | (28) | |
| Graduate university | 1 | (5) | 1 | (6) | |
| First language, n (%) | | | | | 0.001* |
| English | 16 | (84) | 11 | (61) | |
| French | 0 | (0) | 7 | (39) | |
| Other | 3 | (16) | 0 | (0) | |
| Military status, n (%) | | | | | 0.630 |
| Forces | 8 | (42) | 9 | (50) | |
| Reserves | 11 | (58) | 10 | (53) | |
| Rank, n (%) | | | | | <0.001* |
| Junior NCM | 13 | (68) | 5 | (28) | |
| Senior NCM | 0 | (0) | 11 | (61) | |
| Junior officer | 6 | (32) | 2 | (11) | |
| Years of service, med | 5 | (1, 11) | 11 | (9, 14) | 0.005* |
| (IQR) | | | | | |
| Height (cm), med (IQR) | 179 | (173, 188) | 179 | (177, 183) | 0.867 |
| Weight (lbs), med (IQR) | 188 | (170, 200) | 180 | (168, 215) | 0.855 |
| Allergies, n (%) | 6 | (32) | 3 | (17) | 0.447 |
| Medications, n (%) | 5 | (26) | 5 | (29) | >0.994 |
| Coffee/caffeine drinks/ | 1 | (1, 2) | 2 | (1.4, 3) | 0.054 |
| day, med (IQR) | | | | | |
| Alcoholic drinks/week, | 2 | (1, 5) | 2.8 | (1, 8.5) | 0.384 |
| med (IQR) | | | | | |
| Smoke, n (%) | 2 | (11) | 3 | (17) | 0.660 |
| Use drugs in last 6 mo, | 0 | (0) | 1 | (6) | 0.472 |
| n (%) | | | | | |
| Current cold/infection, | 0 | (0) | 3 | (17) | 0.105 |
| n (%) | | | | | |
| Exercise regularly, n (%) | 18 | (95) | 15 | (88) | 0.593 |
| Specific diet, n (%) | 3 | (16) | 2 | (11) | >0.994 |
| Injuries and exposures, | | | | | |
| n (%) | | | | | |
| Concussion | 5 | (26) | 8 | (47) | 0.196 |
| Head impact | 11 | (58) | 9 | (50) | 0.630 |
| Motor vehicle collision | 9 | (47) | 14 | (78) | 0.057 |
| Fall as a child | 6 | (32) | 8 | (44) | 0.420 |
| Physical fight | 15 | (79) | 12 | (67) | 0.476 |
| Blast exposure | 2 | (11) | 18 | (100) | <0.001* |

MBRS = military breachers/range staff. Continuous variables are presented as median (IQR), and categorical variables are presented as n (%).
*p < 0.05.

TABLE 2
Military personnel reported symptoms and health assessment

| | | | |
|---|---|---|---|
| | Non-MBRS | MBRS | P- |
| Variable | (n = 19) | (n = 18) | value |

Reported Symptoms

| | | | | | |
|---|---|---|---|---|---|
| Chronic disease, | 2 | (11) | 1 | (6) | >0.994 |
| n (%) | | | | | |
| Headache | 0 | (0, 2) | 2 | (1, 2.3) | 0.010* |
| Dizziness | 0 | (0, 1) | 1.5 | (0, 2) | 0.043* |
| Vomiting | 0 | (0, 0) | 0 | (0, 0.3) | 0.356 |
| Noise sensitivity | 0 | (0, 0) | 0 | (0, 2) | 0.099 |
| Sleep disturbance | 0 | (0, 0) | 0.5 | (0, 2) | 0.011* |
| Fatigue | 0 | (0, 0) | 1.5 | (0, 2) | 0.002* |
| Irritable | 0 | (0, 0) | 1 | (0, 2.3) | 0.013* |
| Depressed | 0 | (0, 0) | 0 | (0, 1) | 0.003* |
| Frustrated | 0 | (0, 0) | 0.5 | (0, 3) | 0.006* |
| Forgetful | 0 | (0, 0) | 1 | (0, 2) | <0.001* |
| Poor concentration | 0 | (0, 0) | 1 | (0, 2) | <0.001* |
| Taking long to think | 0 | (0, 0) | 1, | (0, 1.3) | 0.003* |
| Blurred vision | 0 | (0, 0) | 0 | (0, 0.3) | 0.137 |
| Double vision | 0 | (0, 0) | 0 | (0, 0.3) | 0.137 |
| Light sensitivity | 0 | (0, 0) | 0 | (0, 1) | 0.030* |
| Restlessness | 0 | (0, 0) | 0 | (0, 1) | 0.007* |
| Impaired | 0 | (0, 0) | 0 | (0, 1) | 0.015* |
| comprehension | | | | | |
| Impaired reasoning | 0 | (0, 0) | 0 | (0, 1) | 0.061 |
| Impaired logic | 0 | (0, 0) | 0 | (0, 0.3) | 0.131 |
| General physical | 5 | (4, 5) | 4 | (3, 4) | 0.002* |
| health | | | | | |
| General mental | 5 | (4, 5) | 4 | (3.8, 4) | 0.037* |
| health | | | | | |

RAND SF-36 Health Survey

| | | | | | |
|---|---|---|---|---|---|
| Physical functioning | 100 | (95, 100) | 98 | (94, 100) | 0.278 |
| Physical limitations | 100 | (100, 100) | 100 | (94, 100) | 0.866 |
| Emotional | 100 | (67, 100) | 100 | (92, 100) | 0.346 |
| limitations | | | | | |
| Energy | 65 | (60, 80) | 48 | (35, 66) | 0.017* |
| Emotional well- | 84 | (68, 88) | 78 | (60, 88) | 0.540 |
| being | | | | | |
| Social functioning | 100 | (75, 100) | 100 | (81, 100) | 0.727 |
| General health | 80 | (65, 95) | 75 | (63, 80) | 0.285 |
| Pain | 90 | (80, 100) | 90 | (79, 93) | 0.187 |

Short Musculoskeletal Function Assessment (SMFA)

| | | | | | |
|---|---|---|---|---|---|
| Function | 34.0 | (34.0, 41.0) | 40.0 | (37.0, 47.0) | 0.018* |
| Bother | 12.0 | (12.0, 17.0) | 15.0 | (12.8, 17.8) | 0.092 |
| Daily activities | 10.0 | (10.0, 10.0) | 10.0 | (10.0, 11.3) | 0.321 |
| Emotional status | 7.0 | (7.0, 13.0) | 11.5 | (9.0, 16.3) | 0.011* |
| Arm and hand | 8 | (8, 8) | 8 | (8, 8) | 0.323 |
| Mobility | 9.0 | (9.0, 10.0) | 9.0 | (9.0, 12.3) | 0.577 |

Rivermead Post Concussion Symptom Questionnaire

| | | | | | |
|---|---|---|---|---|---|
| RPQ3 | 0 | (0, 2.0) | 2.5 | (1.0, 6.0) | 0.004* |
| RPQ13 | 0 | (0, 3.0) | 9.0 | (0.8, 17.8) | 0.001* |
| Somatic | 0 | (0, 0.2) | 0.6 | (0, 1.4) | 0.006* |
| Cognitive | 0 | (0, 0) | 0 | (0, 1.5) | 0.005* |
| Emotional | 0 | (0, 0) | 0 | (0, 1.3) | 0.003* |
| Post-traumatic stress | 0 | (0, 9) | 6 | (0, 11) | 0.106 |
| disorder (PTSD) | | | | | |
| Perceived Stress | 13 | (11, 17) | 16 | (14, 19) | 0.049* |
| Questionnaire (PSQ) | | | | | |

MBRS = military breachers/range staff. Continuous variables are presented as median (IQR), and categorical variables are presented as n (%).
*p < 0.05.

TABLE 3
Military personnel metabolite parameters

| | Non-MBRS | | MBRS | | Direc- | P- |
|---|---|---|---|---|---|---|
| Metabolite | (n = 19) | | (n = 18) | | tion | value |
| Acetic | 36.6 | (29.5, 43.5) | 20.3 | (14.5, 26.4) | ↓ | <0.001 |
| Acid | | | | | | |
| Formate | 58.1 | (55.6, 303.8) | 40.3 | (38.4, 45.5) | ↓ | <0.001 |
| Creatine | 30.1 | (25.7, 38.6) | 20.7 | (17.4, 26.3) | ↓ | <0.001 |
| Acetone | 15.8 | (11.0, 17.6) | 8.7 | (7.4, 9.7) | ↓ | <0.001 |
| Methanol | 42.3 | (28.6, 47.3) | 24.8 | (22.0, 31.5) | ↓ | <0.001 |
| Glutamic | 47.5 | (37.1, 60.0) | 28.2 | (23.2, 37.1) | ↓ | 0.001 |
| Acid | | | | | | |

MBRS = military breachers/range staff. Continuous variables are presented as median (IQR). Metabolite concentrations are in μM.
* p < 0.05.

TABLE 4
ROC curve summary predicting MBRS status

| | AUC | 95% | P- | Cut-off |
|---|---|---|---|---|
| Predictor(s) | (SE) | CI | value | value |
| Individual Metabolites | | | | |
| Acetic Acid | 0.91 (0.05) | 0.80-1.00 | <0.001 | <28.26 |
| Formate | 0.89 (0.06) | 0.77-1.00 | <0.001 | <53.16 |
| Creatine | 0.87 (0.06) | 0.75-0.98 | <0.001 | <22.80 |
| Acetone | 0.90 (0.05) | 0.80-1.00 | <0.001 | <10.77 |
| Methanol | 0.86 (0.06) | 0.74-0.98 | <0.001 | <35.47 |
| Glutamic Acid | 0.82 (0.08) | 0.66-0.97 | 0.001 | <36.90 |
| Parsimonious Combinations | | | | |
| Acetic Acid, Methanol, | 1.00 (0.00) | 1.00-1.00 | <0.001 | — |
| Glutamic Acid | | | | |
| Acetone, Methanol, | 1.00 (0.00) | 1.00-1.00 | <0.001 | — |
| Glutamic Acid | | | | |
| Creatine, Methanol, | 1.00 (0.00) | 1.00-1.00 | <0.001 | — |
| Glutamic Acid | | | | |
| Volatile Organic | | | | |
| Compound Combination | | | | |
| Acetic Acid, Acetone, | 0.98 (0.00) | 0.95-1.00 | <0.001 | — |
| Methanol | | | | |

MBRS = military breachers/range staff. Receiver operating characteristic (ROC) curves were estimated for individual metabolites and continuous outcomes in terms of predicting Breacher status, with area-under-the-curve (AUC) >0.7 considered acceptable. Combinations were created using predicted values from a logistic regression with Breacher status as the outcome and the metabolite combinations as the predictors. Cut-off values were calculated using Youden's index and are presented as μM.

TABLE 5

| Rank | Metabolite | % Importance |
|---|---|---|
| 1 | Acetic acid | 23.7 |
| 2 | Formate | 22.6 |
| 3 | Creatine | 14.8 |
| 4 | Acetone | 14.2 |
| 5 | Methanol | 12.7 |
| 6 | Glutamic acid | 12.0 |

### DISCUSSION

In this study, we performed metabolomics profiling of CAF service members, both MBRS and non-MBRS. Our data confirm that MBRS suffer mTBI symptoms and poorer health. Reduced metabolite parsimonious combinations identified MBRS status with 100% accuracy, and a combination of 3 VOCs with 98% accuracy. Our data suggest that excessive blast exposure in military personnel may be identified with a point-of-care breath analyzer for VOCs, with definitive diagnoses confirmed with laboratory metabolomics profiling of plasma.

Low-level blasts refer to controlled blast exposures that occur during standard training procedures of military personnel. While MBRS are frequently exposed to low-level blasts, exposures are also prevalent for any service member firing artillery, mortars, grenades and/or shoulder-fired weapons. Given the exposure frequency, it is not surprising that up to one quarter of military service members experience post-concussive symptoms. [1,2] Exposure to a 4 psi (28 kPa) blast is considered to be minimally harmful; however, these thresholds were established based on tympanic membrane rupture [5,23] and neglect the cumulative consequences of repetitive exposures. The military doctrine limits blast exposure to 3 psi (21 kPa); however, these values were often exceeded when military personnel exposed to blasts wore pressure gauges. [8,23]

Metabolomics profiling of CAF members included use of both 1H NMR and DI-LC-MS/MS to yield quantitative measurements of 170 metabolites. Feature selection identified the leading 6 metabolites for determining MBRS status, with a 98% classification accuracy. Of the 6 leading metabolites, 5 are related to energy metabolism (acetic acid, formate, creatine, acetone and methanol) and 1 is an excitatory amino acid (glutamic acid). Classification accuracy for determining MBRS status increased to 100% with 3 parsimonious combinations of metabolites, while combing only VOCs resulted in a 98% classification accuracy. Three individual metabolites negatively correlated with Rivermead Post-Concussion Symptoms (acetic acid, creatine and methanol), while RAND Energy and Rivermead early and late symptoms predicted both MBRS status and the 3 metabolite parsimonious combinations. Our metabolite biomarkers appear superior to protein biomarkers for identifying blast exposures.[6]

Acetic acid is absorbed from the gastrointestinal tract and through the lung or formed as a final product of enhanced B-oxidation of fatty acids. Acetic acid is utilized as fuel in extrahepatic tissues and may give rise to the production of ketone bodies as intermediates. Consumption of acetic acid improves glucose tolerance and insulin sensitivity. [24,25] Supplementation with dietary acetic acid is well tolerated, has no adverse side effects, and may improve overall energy metabolism.[26] In brain, acetic acid increases tricarboxylic acid cycle flux and neuronal excitability via glutamate neurotransmission.[27] As MBRS members had significant reductions in plasma acetic acid, and its levels negatively correlated with mTBI symptoms, oral supplementation may aid low energy and neurocognitive symptoms identified by MBRS. As such, administration of acetic acid serves as a treatment of mTBI.

Creatine facilitates ATP homeostasis during energy turnover, and it acts as an antioxidant by attenuating reactive oxygen species.[28] In brain, creatine is also important for energy production via a brain-specific isoform of creatine kinase. Creatine deficiencies result in mental and cognitive derangements, which can be partially attenuated by creatine supplementation.[30] Creatine supplementation is suggested to aid TBI outcome, and clinical trials on military personnel have been encouraged. Indeed, creatine levels in MBRS members negatively correlated with mTBI symptoms. As such, administration of creatine serves as a treatment of mTBI.

Formate is an intermediate in one-carbon (1C) metabolism and is produced in a variety of metabolic reactions within cellular compartments, including folate-dependent (e.g. via serine, glycine, methionine, sarcosine and choline catabolismand folate-independent (e.g. catabolismof tryptophan, methionine salvage, α-oxidation of branched chain fatty acids) reactions. Formate can also be produced by anaerobic fermentation by the gut microbiome. Fermentation of fruits and vegetables by the microbiome can also produce methanol, which is metabolized to formaldehyde and then formate by the liver. Animal studies have demonstrated that spinal cord injuries cause a reduction in intestinal motility and permeability, leading to alterations in intestinal bacterial composition know as gut dysbiosis, whereas TBI causes intestinal bacterial speciation changes as rapidly as 2 h post-trauma.[34] Gut dysbiosis may alter bacterial fermentation-produced circulating methanol and formate levels and thus impact 1C metabolism (including generation of S-adenosylmethionine).[35] Dietary supplementation of 1C sources, including formate, creatine, choline and betaine, may help restore 1C metabolism and possibly ameliorate symptoms of mTBI. As such, administration of 1C sources, including formate, serves as a treatment of mTBI.

Acetone, together with acetoacetate and beta-hydroxybutyrate, are the ketone by-products of fat metabolism in the liver. Acetoacetate is formed from acetyl-CoA, and then beta-oxidized to 3-beta-hydroxbutyrate. When required for energy production, acetoacetate is converted back to acetyl-CoA to be incorporated into the TCA cycle. Decarboxylation of excess acetoacetate produces acetone, which cannot be used for energy production directly and is either exhaled or excreted as waste. However, acetone can also be converted into lactic acid within the liver, which is then subsequently oxidized into pyruvic acid. The latter can also produce acetyl-CoA to be incorporated into the TCA cycle. The decreased plasma acetone levels measured in MBRS members may reflect less acetoacetate decarboxylation and/or greater acetone conversion to pyruvic acid, to compensate energy deficits. In addition, elevating plasma ketones via dietary manipulation (i.e., carbohydrate restriction) may improve blast-induced symptoms.[37] Indeed, ketones are actively transported into brain via monocarboxylate transporters, and up to two-thirds of brain metabolism can be fueled by ketones. As such, low carbohydrate diet serves as a treatment of mTBI.

Glutamic acid is a non-essential amino acid that is a major mediator of excitatory signals in the brain and is involved in most aspects of normal brain function including cognition, memory and learning. Measurements of glutamic acid in plasma are generally thought to reflect brain levels, as there are no glutamate degrading enzymes and regulation of glutamic acid levels is controlled via cellular release and cellular uptake.[39,40] High plasma glutamic acid levels are associated with acute/sub-acute TBI, as well as anxiety, autism, bipolar disorder, depression, impulsivity and stroke. Low plasma glutamic acid levels are often attributed to ammonia toxicity, and more recently, to Parkinson's disease.[41] The low levels of glutamic acid found in plasma from MBRS members may represent a relative exhaustion of glutamate production after chronic low-level blast exposure and separates itself from subacute mechanical TBI.

There is significant novelty and advantageous use of the data presented herein. We report that a small number of metabolites can accurately determine whom has been chronically exposed to low-level blasts, and that the measured decreases in plasma metabolites correlate with increased mTBI symptoms. Furthermore, a combination of VOCs can be accurately measured with portable, hand-held breathalyzers, and can be used as a point-of-care monitoring devices for service members in both training and theatre. A laboratory quantitative test can be easily developed that would require analysis of a blood sample with either 1H NMR or quantitative mass spectrometry. Indeed, the latter approach is currently under clinical testing for concussion diagnostics. As the metabolite profile for chronic blast injury is unique, it is possible that metabolite signatures will be useful for separating various forms of trauma, including blast, mechanical, neurochemical and psychological.

In summary, we report a unique metabolite signature in military personnel suffering from mTBI symptoms and poorer health exposed to repetitive low-level blasts. Reduced plasma metabolite combinations associated with energy metabolism and an excitatory amino acid neurotransmitter identified MBRS status with 100% accuracy. A combination of 3 VOCs identified MBRS status with 98% accuracy. Excessive blast exposure in military personnel may be identified with a point-of-care breath analyzer for VOCs, with definitive diagnoses confirmed with metabolomics profiling of plasma. A device for collecting and analyzing vapor condensate (VC) sample can be used to collect and measure the 3 VOCs. The metabolite biomarkers for blast exposure identified here may aid blast injury surveillance and care of military personnel. Oral supplementation with both acetic acid, creatine and 1C sources, as well as carbohydrate restriction (i.e., ketosis), may alleviate some blast-induced symptoms related to brain energy metabolism.

### REFERENCES

1. Garber B G, Rusu C, Zamorski M A. Deployment-related mild traumatic brain injury, mental health problems, and post-concussive symptoms in Canadian Armed Forces personnel. BMC Psychiatry. 2014; 14:325.
2. Terrio H, Brenner L A, Ivins B J, et al. Traumatic brain injury screening: preliminary findings in a US Army Brigade Combat Team. J Head Trauma Rehabil. 2009; 24(1): 14-23.
3. Bryden D W, Tilghman J I, Hinds S R, 2nd. Blast-Related Traumatic Brain Injury: Current Concepts and Research Considerations. J Exp Neurosci. 2019; 13:1179069519872213.
4. Pathophysiology of Blast Injury and Overview of Experimental Data. In: Committee on Gulf War and Health: Long-Term Effects of Blast Exposures; Board on the Health of Select Populations; Institute of Medicine. Vol 9. Washington, DC: National Academies Press; 2014.
5. Carr W, Stone J R, Walilko T, et al. Repeated Low-Level Blast Exposure: A Descriptive Human Subjects Study. Mil Med. 2016;181(5 Suppl):28-39.
6. Kamimori G H, LaValle C R, Eonta S E, Carr W, Tate C, Wang K K W. Longitudinal Investigation of Neurotrauma Serum Biomarkers, Behavioral Characterization, and Brain Imaging in Soldiers Following Repeated Low-Level Blast Exposure (New Zealand Breacher Study). Mil Med. 2018;183(suppl_1):28-33.
7 Vartanian O, Tenn C, Rhind S G, et al. Blast in Context: The Neuropsychological and Neurocognitive Effects of Long-Term Occupational Exposure to Repeated Low-Level Explosives on Canadian Armed Forces′ Breaching Instructors and Range Staff. Front Neurol. 2020; 11:588531.
8. Nakashima A, Vartanian O, Rhind S G, King K, Tenn C, Jetly C R. Repeated Occupational Exposure to Low-level Blast in the Canadian Armed Forces: Effects on Hearing, Balance, and Ataxia. Mil Med. 2021.
9 Mellor S G, Cooper G J. Analysis of 828 servicemen killed or injured by explosion in Northern Ireland 1970-84: the Hostile Action Casualty System. Br J Surg. 1989; 76(10): 1006-1010.
10. Cernak I, Savic J, Malicevic Z, et al. Involvement of the central nervous system in the general response to pulmonary blast injury. J Trauma. 1996;40(3 Suppl):S100-104.
11. Cernak I, Radosevic P, Malicevic Z, Savic J. Experimental magnesium depletion in adult rabbits caused by blast overpressure. Magnes Res. 1995; 8(3):249-259.
12. Readnower R D, Chavko M, Adeeb S, et al. Increase in blood-brain barrier permeability, oxidative stress, and activated microglia in a rat model of blast-induced traumatic brain injury. J Neurosci Res. 2010; 88(16):3530-3539.
13. Cernak I, Merkle A C, Koliatsos V E, et al. The pathobiology of blast injuries and blast-induced neurotrauma as identified using a new experimental model of injury in mice. Neurobiol Dis. 2011; 41(2):538-551.
14. Kaur C, Singh J, Lim M K, Ng B L, Ling E A. Macrophages/microglia as ‘sensors’ of injury in the pineal gland of rats following a non-penetrative blast. Neurosci Res. 1997; 27(4):317-322.
15. Kaur C, Singh J, Lim M K, Ng B L, Yap E P, Ling E A. The response of neurons and microglia to blast injury in the rat brain. Neuropathol Appl Neurobiol. 1995; 21(5):369-377.
16. Saljo A, Bao F, Hamberger A, Haglid K G, Hansson H A. Exposure to short-lasting impulse noise causes microglial and astroglial cell activation in the adult rat brain. Pathophysiology. 2001; 8(2):105-111.
17. Bujak R, Struck-Lewicka W, Markuszewski M J, Kaliszan R. Metabolomics for laboratory diagnostics. J Pharm Biomed Anal. 2015; 113:108-120.
18. Daley M, Dekaban G, Bartha R, et al. Metabolomics profiling of concussion in adolescent male hockey players: a novel diagnostic method. Metabolomics. 2016; 12(12): 185.
19. Tang C, Garreau D, von Luxburg U. When do random forests fail? Proceedings of the 32nd International Conference on Neural Information Processing Systems. 2018; December 2018:2987-2997.
20. van der Maaten L, Hinton G. Visualizing data using t-SNE. J Mach Learn Res. 2008; 9(11):2579-2605.
21. Wickham H. ggplot2: Elegant Graphics for Data Analysis. Springer Publishing Company, Incorporated; 2009.
22. Belanger H G, Bowling F, Yao E F. Low-Level Blast Exposure in Humans A Systematic Review of Acute and Chronic Effects. J Spec Oper Med. 2020; 20(1):87-93.
23. Kamimori G H, Reilly L A, LaValle C R, Olaghere Da Silva U B. Occupational overpressure exposure of breachers and military personnel. Shock Waves. 2017; 27(6):837-847.
24. Mitrou P, Petsiou E, Papakonstantinou E, et al. The role of acetic acid on glucose uptake and blood flow rates in the skeletal muscle in humans with impaired glucose tolerance. Eur J Clin Nutr. 2015; 69(6): 734-739.
25. Yamashita H. Biological Function of Acetic Acid-Improvement in Obesity and Glucose Tolerance by Acetic Acid in Type 2 Diabetic Rats. Crit Rev Food Sci Nutr. 2016;56 Suppl 1:S171-175.
26. Valdes D S, So D, Gill P A, Kellow N J. Effect of Dietary Acetic Acid Supplementation on Plasma Glucose, Lipid Profiles, and Body Mass Index in Human Adults: A Systematic Review and Meta-analysis. J Acad Nutr Diet. 2021; 121(5): 895-914.
27. Jakkamsetti V, Marin-Valencia I, Ma Q, et al. Brain metabolism modulates neuronal excitability in a mouse model of pyruvate dehydrogenase deficiency. Sci Transl Med. 2019;11(480).
28. Roschel H, Gualano B, Ostojic S M, Rawson E S. Creatine Supplementation and Brain Health. Nutrients. 2021;13(2).
29. Andres R H, Ducray A D, Schlattner U, Wallimann T, Widmer H R. Functions and effects of creatine in the central nervous system. Brain Res Bull. 2008; 76(4): 329-343.
30. Gualano B, Artioli G G, Poortmans J R, Lancha Junior A H. Exploring the therapeutic role of creatine supplementation. Amino Acids. 2010; 38(1):31-44.
31. Nutrition and Traumatic Brain Injury: Improving Acute and Subacute Health Outcomes in Military Personnel; Institute of Medicine. Washington, DC: The National Academies Press; 2011.
32. Lamarre S G, Morrow G, Macmillan L, Brosnan M E, Brosnan J T. Formate: an essential metabolite, a biomarker, or more? Clin Chem Lab Med. 2013; 51(3):571-578.
33. Pietzke M, Meiser J, Vazquez A. Formate metabolism in health and disease. Mol Metab. 2020; 33:23-37.
34. Rice M W, Pandya J D, Shear D A. Gut Microbiota as a Therapeutic Target to Ameliorate the Biochemical, Neuroanatomical, and Behavioral Effects of Traumatic Brain Injuries. Front Neurol. 2019; 10:875.
35. Suh E, Choi S-W, Friso S. One-Carbon Metabolism: An Unsung Hero for Healthy Aging. 2016.
36. Akram M. A focused review of the role of ketone bodies in health and disease. J Med Food. 2013; 16(11): 965-967.
37. Yang H, Shan W, Zhu F, Wu J, Wang Q. Ketone Bodies in Neurological Diseases: Focus on Neuroprotection and Underlying Mechanisms. Front Neurol. 2019; 10:585.
38. Zhou Y, Danbolt N C. Glutamate as a neurotransmitter in the healthy brain. J Neural Transm (Vienna). 2014; 121(8):799-817.
39. Teichberg V I, Cohen-Kashi-Malina K, Cooper I, Zlotnik A. Homeostasis of glutamate in brain fluids: an accelerated brain-to-blood efflux of excess glutamate is produced by blood glutamate scavenging and offers protection from neuropathologies. Neuroscience. 2009; 158(1):301-308.
40. Vandenberg R J, Ryan R M. Mechanisms of glutamate transport. Physiol Rev. 2013; 93(4): 1621-1657.
41. Fiandaca M S, Gross T J, Johnson T M, et al. Potential Metabolomic Linkage in Blood between Parkinson's Disease and Traumatic Brain Injury. Metabolites. 2018;8(3).
42. Miller M R, Robinson M, Bartha R, et al. Concussion Acutely Decreases Plasma Glycerophospholipids in Adolescent Male Athletes. J Neurotrauma. 2021; 38(12): 1608-1614.

Through the embodiments that are illustrated and described, the currently contemplated best mode of making and using the disclosure is described. Without further elaboration, it is believed that one of ordinary skill in the art can, based on the description presented herein, utilize the present disclosure to the full extent. All publications cited herein are incorporated by reference.

Although the description above contains many specificities, these should not be construed as limiting the scope of the disclosure, but as merely providing illustrations of some of the presently embodiments of this disclosure.

1. A method of diagnosing mild traumatic brain injury (mTBI) in a subject comprising:
(a) obtaining a test sample from the subject;
(b) quantitatively or semi-quantitatively measuring levels of a biomarker in the test sample;
(c) comparing the levels of the biomarker in the test sample with a known normal reference level of the biomarker,
wherein a decrease in the level of the biomarker in the test sample relative to the known normal reference level of the biomarker is indicative of mTBI diagnosis in the subject, the biomarker being one of acetic acid, formate, creatine, acetone, methanol or glutamic acid or a combination of two, three, four, five or six of acetic acid, formate, creatine, acetone, methanol and glutamic acid, and
(d) treating the subject for mTBI only when the levels of the biomarker in the test sample is decreased relative to the known normal reference.

2. The method of claim 1, wherein the method further comprises obtaining one or more recovery samples from the subject during the subject's treatment for mTBI, wherein an increase in the levels of the biomarker in the one or more recovery samples relative to the levels of the biomarker obtained in the test sample is indicative of a normalization of the subject.

3. (canceled)

4. The method of claim 1, wherein said treatment includes administering to the subject acetic acid, a source of acetic acid, creatine, a source of creatine, one-carbon (1C) sources or carbohydrate restriction, glutamic acid, a source of glutamic acid, or a combination thereof.

5. The method of claim 1, wherein the biomarker is one of acetic acid, formate, creatine, acetone, methanol or glutamic acid.

6. The method of claim 1, wherein the sample is blood and wherein the biomarker is acetic acid and the known normal reference level is about 29 μM, or the biomarker is formate and the known normal reference level is about 54 μM, or the biomarker is creatine and the known normal reference level is about 23 μM, or the biomarker is acetone and the known normal reference level is about 11 μM, or the biomarker is methanol and the known normal reference level is about 36 μM, or the biomarker is glutamic acid and the known normal reference level is about 37 μM.

7. The method of claim 1, wherein the biomarker is a combination of acetone, methanol and glutamic acid.

8. The method of claim 1, wherein the biomarker is a combination of creatine, methanol, and glutamic acid.

9. The method of claim 1, wherein the biomarker is a combination of acetic acid, acetone and methanol.

10. The method of claim 1, wherein the sample is a blood sample, a plasma sample, a serum sample, a capillary sample, a sweat sample, a tear sample, an exhale breath condensate sample, a urine sample, a saliva sample, or a combination thereof.

11. The method of claim 1, wherein the mTBI is primary blast in blast-induced traumatic brain injury or concussion.

12. A mild traumatic brain injury (mTBI) diagnostic apparatus, the mTBI diagnostic apparatus including a non-transitory computer readable storage medium and a computer program mechanism embedded therein, the computer program mechanism comprising executable instructions for performing a method of diagnosing mTBI in a subject, said executable instructions comprising:
(a) receiving quantitative or semi-quantitative levels of a biomarker in a test sample of the subject;
(b) comparing the quantitative or semi-quantitative levels of the biomarker in the test sample of the subject to known normal reference levels of the biomarker; and
(c) providing a mTBI positive signal when there is a decrease in the level of the biomarker the test sample relative to the known normal reference, the biomarker being one of acetic acid, formate, creatine, acetone, methanol or glutamic acid or a combination of two, three, four, five or six of acetic acid, formate, creatine, acetone, methanol, and glutamic acid.

13. The apparatus of claim 12, wherein the biomarker is one of acetic acid, formate, creatine, acetone, methanol, or glutamic acid.

14. The apparatus of claim 12, wherein the sample is blood and wherein the biomarker is acetic acid and the known normal reference level is about 29 μM, or the biomarker is formate and the known normal reference level is about 54 μM, or the biomarker is creatine and the known normal reference level is about 23 μM, or the biomarker is acetone and the known normal reference level is about 11 μM, or the biomarker is methanol and the known normal reference level is about 36 μM, or the biomarker is glutamic acid and the known normal reference level is about 37 μM.

15. The apparatus of claim 12, wherein the biomarker is a combination of acetone, methanol, and glutamic acid.

16. The apparatus of claim 12, wherein the biomarker is a combination of creatine, methanol, and glutamic acid.

17. The apparatus of claim 12, wherein the biomarker is a combination of acetic acid, acetone, and methanol.

18. The apparatus of claim 12, wherein the biomarker is one of acetic acid, acetone or methanol, or a combination of two or more of acetic acid, acetone and methanol, and the apparatus is a breathalyzer, a portable mass spectrometer (MS) or a collection device for laboratory MS measurement.

19. The apparatus of claim 12, wherein the mTBI is primary blast in blast-induced traumatic brain injury.

20. The apparatus of claim 12, wherein the mTBI is concussion.

21-26. (canceled)

27. A method of treating a subject suffering mild traumatic brain injury (mTBI), wherein the method comprises administering to the subject one or a combination of two or more of acetic acid, a source of acetic acid, creatine, a source of creatine, one-carbon nutrients, a low carbohydrate diet, glutamic acid, and a source of glutamic acid.

28. (canceled)
